(19) United States
(12) Patent Application Publication
(10) Pub. No.: US 2006/0025667 A1
(43) Pub. Date: Feb. 2, 2006

(54) Method for tumor perfusion assessment in clinical trials using dynamic contrast enhanced MRI

(76) Inventor: Edward Ashton, Webster, NY (US)

Correspondence Address: BLANK ROME LLP, 600 NEW HAMPSHIRE AVENUE, N.W., WASHINGTON, DC, 20037, US

(21) Appl. No.: 10/901,160

(22) Filed: Jul. 29, 2004

(51) Int. Cl. A61B 5/05

(52) U.S. Cl. 600/407

(57) ABSTRACT

In a clinical trial using dceMRI, the assessment of tumor perfusion has problems of noise and reproducibility. To address those problems, an end-to-end method develops and enforces a standard imaging protocol, ensures site compliance both by pre-qualification and throughout the trial, ensures that the scanners function properly both at the outset and throughout the trial, develops an analysis process with automation and quality control to prevent human error, and provides analysis software to perform the assessment and to provide an electronic audit trail.

### FIELD OF THE INVENTION

The present invention is directed to a method for tumor perfusion assessment and more particularly to such a method in which the most significant factors driving reproducibility are addressed.

### DESCRIPTION OF RELATED ART

Dynamic contrast enhanced Magnetic Resonance Imaging (dceMRI) has demonstrated considerable utility in both diagnosing and evaluating the progression and response to treatment of malignant tumors. By making use of a two-compartment model, with one compartment representing blood and the other abnormal extra-vascular extra-cellular space (EES), the observed uptake curves in tissue and blood can be used to estimate various physiological parameters relating to tumor vascularity.

In a clinical trial setting it is critical to be able to accurately measure the change in these parameters over time due to disease progression or response to therapy. Measurement reproducibility must therefore be of primary concern when designing a system for perfusion assessment in clinical trials. Reproducibility can be adversely impacted by random noise introduced at many stages in the measurement process, from data acquisition to final report generation.

### SUMMARY OF THE INVENTION

It is therefore an object of the invention to design an end-to-end analysis technique for tumor perfusion assessment which would provide maximum measurement reproducibility through the elimination of as many of these noise sources as possible.

To achieve the above and other objects, the present invention addresses the factors driving reproducibility: namely, site compliance, analysis software, analysis process, scanners, and imaging protocol. The present invention addresses each of the factors in the following ways.

Site compliance: The present invention addresses site compliance through pre-qualification of site equipment and personnel, face-to-face training for all participating technicians, and continuous feedback to sites on compliance and quality

Analysis software: The software performs automated warp-based registration to align time points and semi-automated tumor margin ID using geometrically constrained region growth. It then performs automated AIF identification (AIF is the arterial input function, or the concentration of contrast agent in an artery that feeds the tissue of interest) and automated parameter calculation using the Tofts or Lee model. Finally, it forms a complete electronic audit trail compliant with Food and Drug Administration regulations (21 C.F.R. part 11).

Analysis process: An automated, script-driven analysis process prevents human error in data handling. Multiple QA/QC (quality assurance/quality control) steps minimize analyst or reader error. A rigorous software development process and version control system prevent altered results through software changes.

Scanners: The scanners are checked for proper functioning by scanning a phantom and analyzing the results. The following steps are carried out: developing linearity, volume and T2 phantoms; scanning and analyzing during site qualification; scanning and analyzing monthly throughout the trial; and requiring maintenance for any failed scanners before proceeding.

Imaging protocol: Imaging sites differ in their preferred dceMRI protocols, making cross-site comparability difficult. Examples of such differences include quiet breathing vs. breath hold, coverage vs. signal-to-noise ratio (SNR) vs. temporal resolution, and differences in dose and rate of contrast injection. Careful development and enforcement of a standard protocol is crucial for cross-site comparability.

This system has been tested using dceMRI data taken from both human and canine subjects. The statistic of interest in both experiments was coefficient of variability for multiple measurements of a single data set by multiple operators. In the animal experiment the rate transfer constant between plasma and EES (K^{trans}) for three subjects over three time points was measured by four independent analysts (a total of 36 analyses) using both manual and automated AIF identification. Using manual AIFs, coefficients of variability ranged from 3.1% to 39.2%, with a mean of 20.1% and a median value of 21.5%. For the nine automated plasma identifications, coefficients of variability ranged from 3.1% to 11.8%, with a mean of 6.7% and a median value of 6.2%. In the human experiment, K^{trans} was measured for 12 subjects over two time points (24 image data sets measured once each by four independent operators, for a total of 96 analyses). Using manual AIFs, coefficients of variability ranged from 1% to 43%, with a mean of 13.1% and a median value of 11%. Using automated AIFs, coefficients of variability ranged from 1% to 38%, with a mean of 9.8% and a median value of 6%. Note that the variability results for humans using automated AIFs are very similar to those seen in the canine experiment, while the variability results for humans using manual AIFs are significantly better than those for canines. This is as expected, since the smaller vessel sizes and significantly higher blood velocity in canines make identification of arterial signal that is uncorrupted by artifacts much more difficult in canines than in humans.

### BRIEF DESCRIPTION OF THE DRAWINGS

A preferred embodiment of the present invention will be set forth in detail with reference to the drawings, in which:

FIG. 1 is a conceptual diagram of the factors driving reproducibility;

FIG. 2 is a flow chart showing a technique used in the preferred embodiment to ensure site compliance;

FIG. 3 shows part of a questionnaire used in conjunction with the technique of FIG. 2;

FIG. 4 is a flow chart showing the operation of analysis software in the preferred embodiment;

FIGS. 5A and 5B show steps in the automated warp-based registration to align time points as carried out in the operation of FIG. 4;

FIGS. 6A-6D show steps in the semi-automated tumor-margin identification as carried out in the operation of FIG. 4;

FIG. 7A shows a plot of automated AIF identification carried out in the operation of FIG. 4;

FIG. 7B shows automated parameter calculation carried out in the operation of FIG. 4;

FIG. 8 shows a portion of a Part 11 compliant electronic audit trail produced in the operation of FIG. 4;

FIG. 9 shows a flow chart of an image acquisition and analysis process;

FIG. 10 shows a flow chart of a software validation process carried out in conjunction with the process of FIG. 9;

FIG. 11 shows a flow chart of scanner analysis and maintenance; and

FIG. 12 shows examples of acceptable and unacceptable scanner outputs produced in the scanner analysis and maintenance of FIG. 11.

### DETAILED DESCRIPTION OF THE PREFERRED EMBODIMENT

A preferred embodiment of the present invention will now be set forth in detail with reference to the drawings.

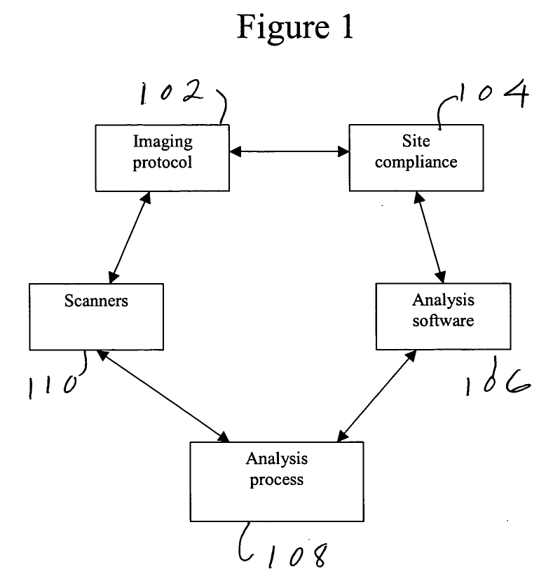

As shown in FIG. 1, five factors drive reproducibility: the imaging protocol 102, site compliance 104, the analysis software 106, the analysis process 108, and the calibration and maintenance of the scanners 110. Each of the five factors will be described below. It will be seen that while the five factors are shown in FIG. 1 as discrete, they are interrelated. It will also be understood that they do not have to be considered in the order in which they are disclosed below.

Imaging Protocol

As noted above, imaging sites differ in their preferred dceMRI protocols, making cross-site comparability difficult. Examples of such differences include quiet breathing vs. breath hold, coverage vs. signal-to-noise ratio (SNR) vs. temporal resolution, and differences in dose and rate of contrast injection.

It is therefore a part of the preferred embodiment to develop and enforce a standard protocol for cross-site compatibility. The specifics of the standard protocol are less important than that the protocol be standard across all sites; therefore, any of the above options, or other options, can be used.

Once the standard protocol has been decided, it can be set forth in an operations guide, to be given to all of the sites and used during the on-site training that is part of site compliance.

Site Compliance

It is not enough to develop an imaging protocol, analysis software, or the like. Instead, it should be ensured that each site complies with the protocols developed.

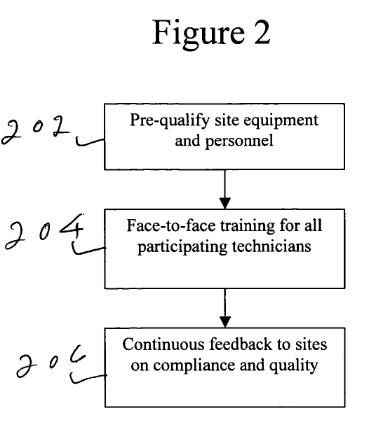

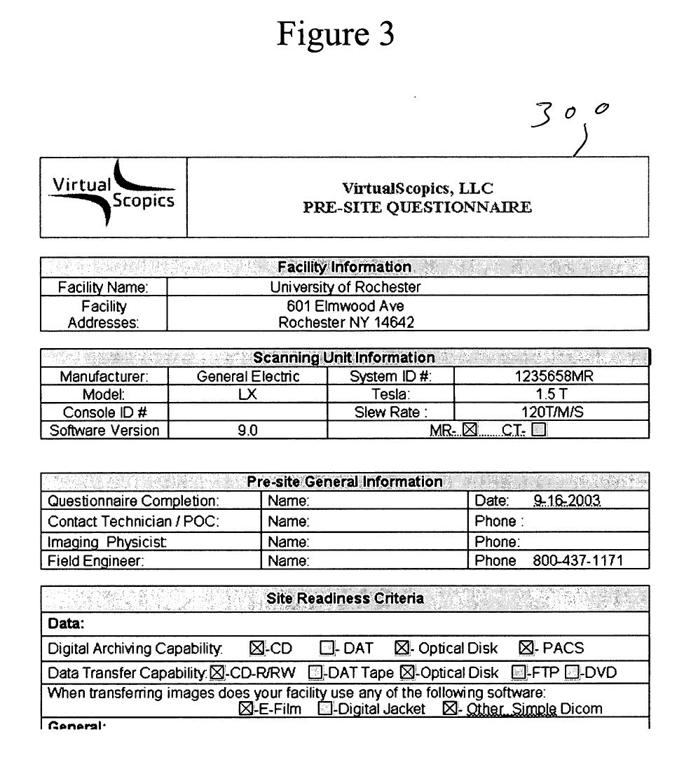

FIG. 2 shows a flow chart of steps to ensure site compliance. In step 202, the equipment and personnel at a site are pre-qualified. Pre-qualification can be performed through a pre-site questionnaire such as that shown partially in FIG. 3 as 300. In step 204, face-to-face training is performed for all participating technicians, as well as for any other persons for whom it may be appropriate. Such face-to-face training may be performed periodically as needed and includes such matters as the imaging protocol and the use of the analysis software. In step 206, continuous feedback is provided to the site on compliance and quality. Such continuous feedback ensures that the site will not drift from the protocols originally implemented.

Analysis Software

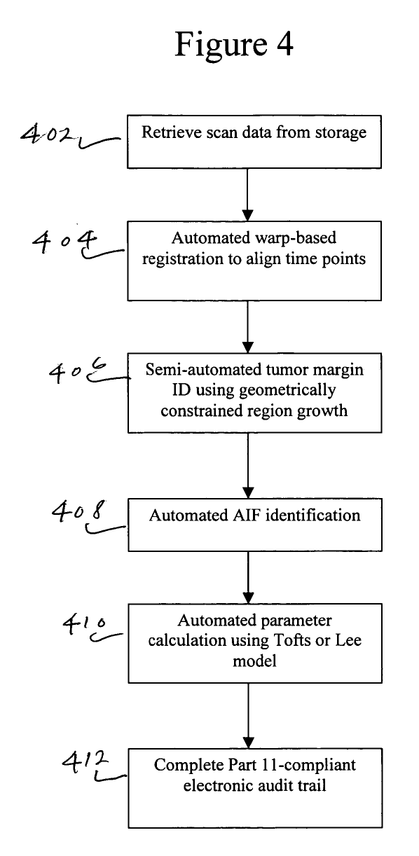

Software is provided as part of the preferred embodiment to identify the AIF and calculate the parameters relating to tumor vascularity. The software will be described with reference to FIGS. 4-8.

According to the flow chart of FIG. 4, first, in step 402, the scan data are retrieved from storage. Alternatively, they could be processed in real time.

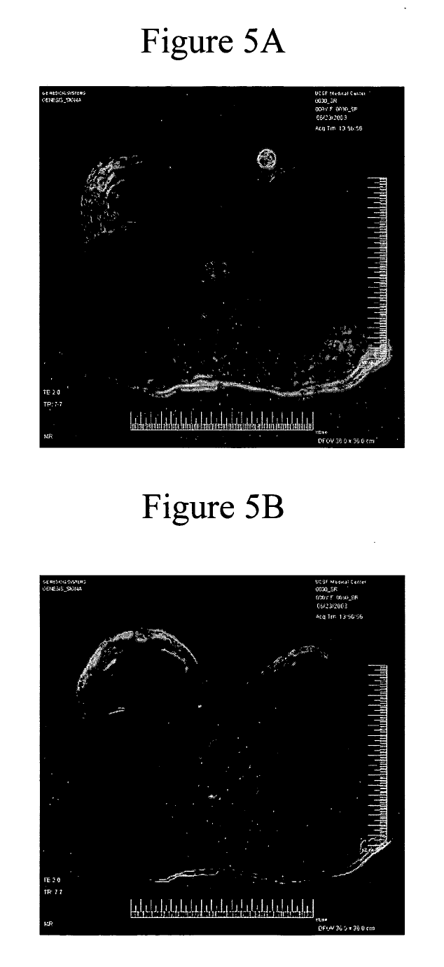

In step 404, an automated warp-based registration is performed to align time points. For example, as shown in FIG. 5A, a series of images are superimposed. A warp-based registration is performed to register the images to produce the image of FIG. 5B.

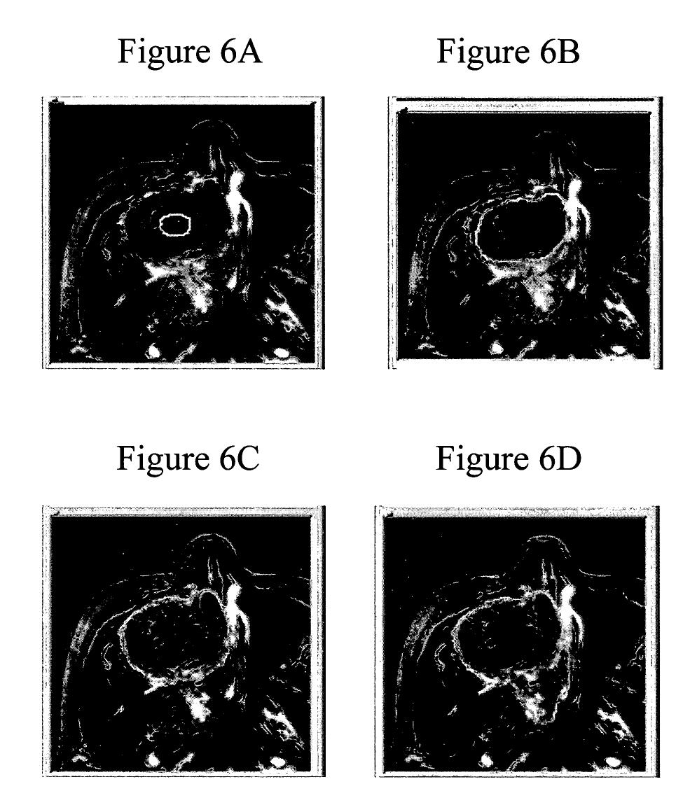

In step 406, a semi-automated tumor margin identification is performed using geometrically constrained region growth. For example, FIGS. 6A-6D show successive stages in such an identification. FIG. 6A shows a seed region drawn by a user in the tumor, which is then grown to identify the tumor margin. FIGS. 6B-6D show successively grown regions that provide successive approximations of the tumor margin. The process is iterated until a stable result is achieved.

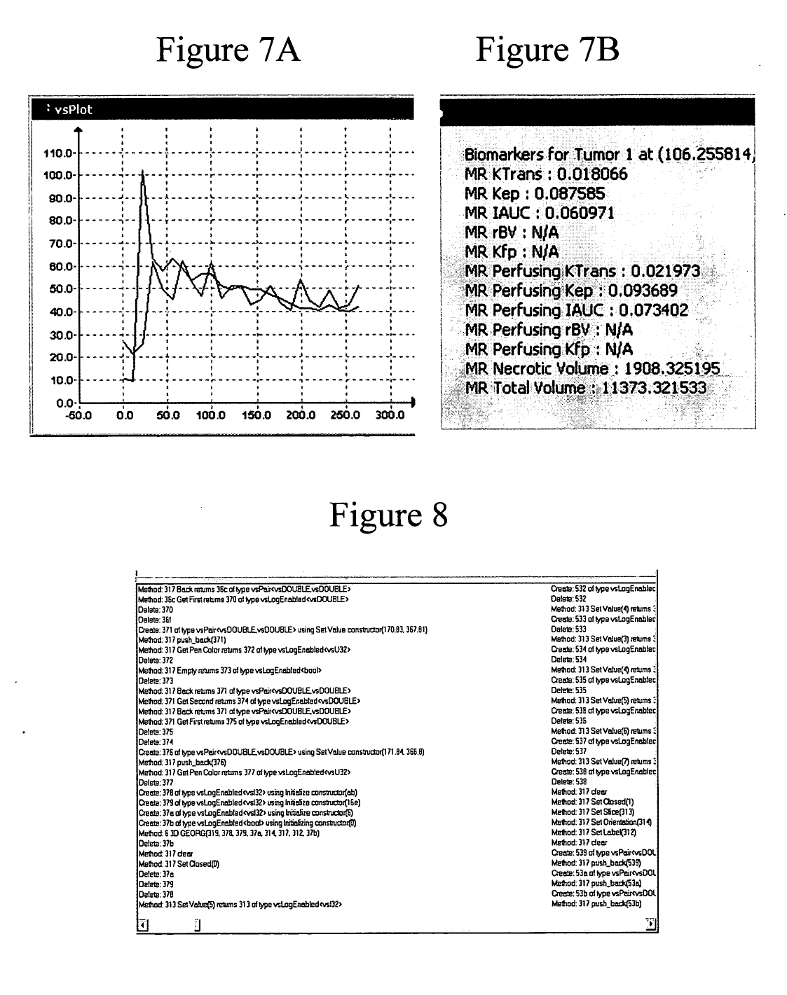

In step 408, the AIF is automatically identified. FIG. 7A shows an example of a result.

In step 410, the parameters relating to tumor vascularity are automatically calculated, using an appropriate technique such as the Tofts or Lee model. FIG. 7B shows an example of results.

In step 412, an electronic audit trail compliant with 21 C.F.R. part 11 is completed and stored for later use. An example is shown in FIG. 8.

Analysis Process

The analysis process incorporates an automated, script-driven process to prevent human error in data handling. Multiple QA/QC (quality assurance/quality control) steps minimize analyst or reader error. A rigorous software development process and version control system prevent altered results through software changes.

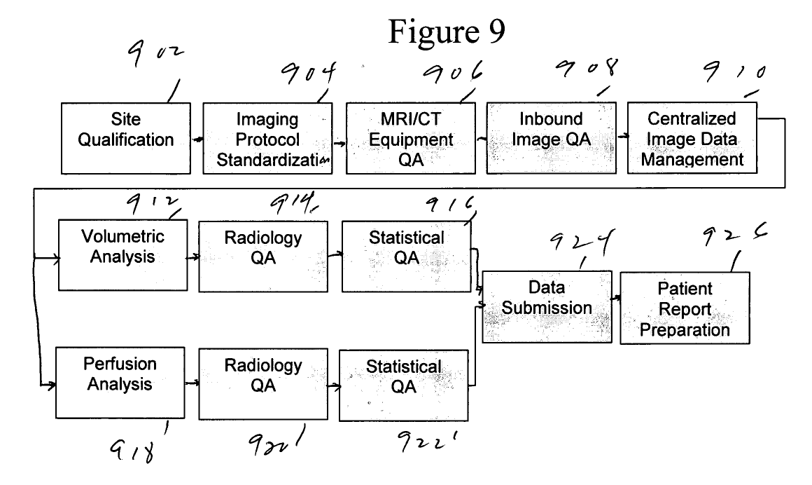

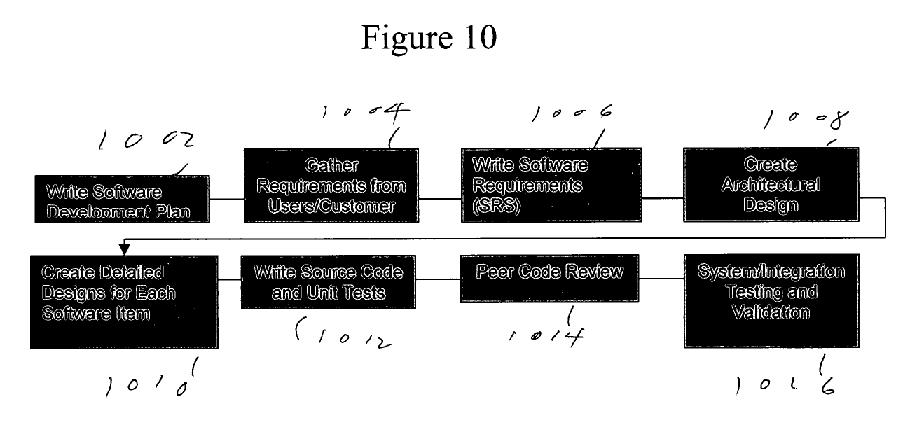

An image acquisition analysis process is shown in FIG. 9. A software validation process is shown in FIG. 10.

In FIG. 9, step 902, a site qualification is performed, as described above. In step 904, an imaging protocol is standardized, also as described above. In step 906, quality assurance is performed on the MRI/CT equipment, in a manner to be described below. In step 908, quality assurance is performed on inbound images. In step 910, centralized image data management, e.g., maintenance and backup of a centralized image server, is performed.

Once the image data are available on a centralized image server, the process splits into two branches that can be carried out independently of each other. In the first branch, in step 912, a volumetric analysis is performed on the image data to determine the tumor volume. Radiology QA and statistical QA a performed in steps 914 and 916. In the second branch, a perfusion analysis is performed in step 918 to assess tumor perfusion. Radiology QA and statistical QA are performed in steps 920 and 922. When the results from the two branches are available, the data are submitted in step 924, so that a patient report can be prepared in step 926.

The software validation process will now be described. In step 1002, the software development plan is written. In step 1004, requirements are gathered from users/customers. In step 1006, software requirements are written. In step 1008, an architectural design is created for the software. In step 1010, detailed designs are created for each software item. In step 1012, the source code and unit tests are written; they are peer reviewed in step 1014. In step 1014, the system is tested and validated.

Scanner Quality Assurance

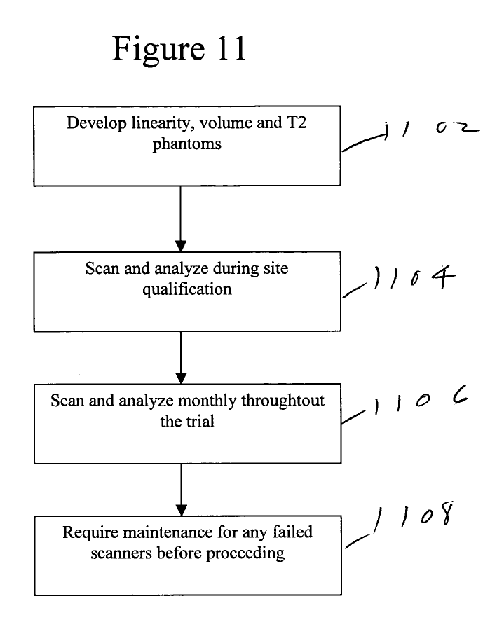

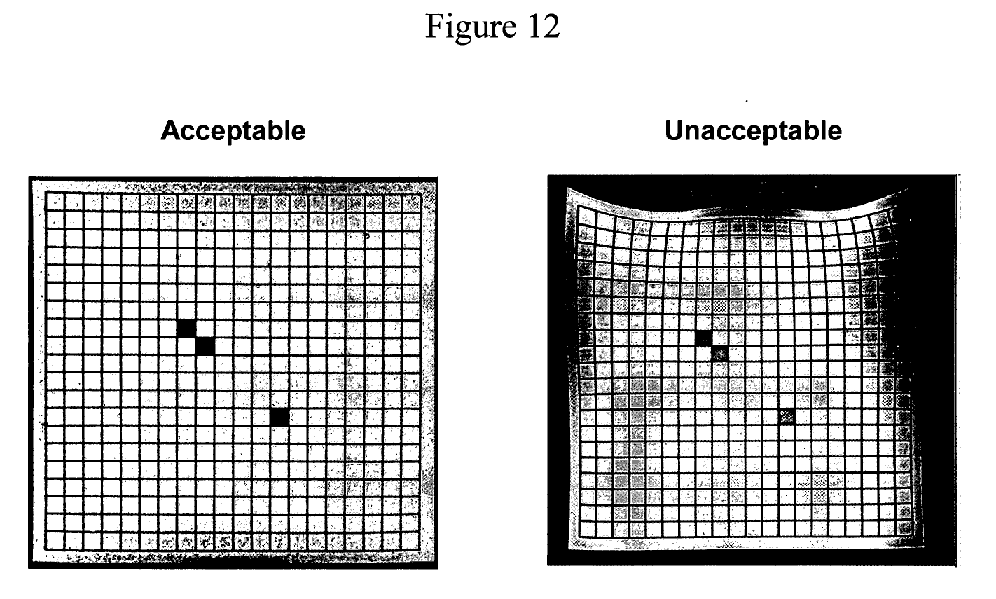

Scanner quality assurance will be described with reference to FIGS. 11 and 12. In step 1102, linearity, volume, and T2 phantoms are developed. In step 1104, the phantoms are scanned, and the resulting image data are analyzed, during site qualification. FIG. 12 shows examples of acceptable (left) and unacceptable (right) image data from a phantom. In step 1106, the phantoms are again scanned, and the resulting image data are again analyzed, on a routine basis (e.g., monthly) throughout the trial. In step 1108, maintenance is performed on any failed scanners before any process that uses them proceeds.

It will be seen from the above that an end-to-end technique has been developed for tumor perfusion analysis in which the various sources of noise have been addressed. While various elements or steps in the technique may be familiar to those skilled in the art, the end-to-end technique itself is believed to be novel.

While a preferred embodiment has been set forth in detail above, those skilled in the art who have reviewed the present disclosure will readily appreciate that other embodiments can be realized within the scope of the invention. For instance, the examples given above for the pre-qualification questionnaire and the like are illustrative rather than limiting. Also, the order in which the factors are described does not limit the order in which the various steps in the end-to-end technique can be carried out. Moreover, while certain U.S. regulations have been cited, the invention can readily be adapted to conform to other countries' regulations. Therefore, the present invention should be construed as limited only by the appended claims.

What is claimed is:

1. A method for providing reproducible measurements of parameters relating to vascularity of a tumor in a patient during a clinical trial and for reducing or eliminating effects of noise on the measurements of the parameters, the method comprising:
(a) developing a standard imaging protocol for use at a plurality of sites, each of the plurality of sites having at least one scanner on which the imaging protocol is to be implemented;
(b) ensuring that each of the plurality of sites complies with the standard imaging protocol;
(c) ensuring that the at least one scanner at each of the plurality of sites is operating correctly;
(d) developing an automated process for analyzing image data taken from the tumor to provide the reproducible measurements;
(e) taking the image data from the tumor using a scanner at one of the plurality of sites; and
(f) determining the reproducible measurements from the image data in step (e), using the automated process of step (d).

2. The method of claim 1, wherein step (e) is performed through dynamic contrast enhanced magnetic resonance imaging.

3. The method of claim 2, wherein the standard imaging protocol of step (a) specifies at least one of the following: the patient's breathing, a dose and rate of contrast injection into the patient, and an optimization of one of coverage, signal-to-noise ratio, and temporal resolution.

4. The method of claim 3, wherein step (b) comprises face-to-face training of participating technicians at each of the plurality of sites in the standard imaging protocol.

5. The method of claim 2, wherein step (b) comprises:
(i) pre-qualifying each of the plurality of sites to determine whether each of the plurality of sites is capable of implementing the standard imaging protocol;
(ii) face-to-face training of participating technicians at each of the plurality of sites in the standard imaging protocol; and
(iii) providing feedback to each of the plurality of sites on compliance with the standard imaging protocol and quality of image data.

6. The method of claim 5, wherein step (b)(iii) is performed a plurality of times for each of the plurality of sites throughout the clinical trial.

7. The method of claim 2, wherein step (c) comprises:
(i) providing at least one phantom;
(ii) imaging the at least one phantom in the at least one scanner at each of the plurality of sites;
(iii) determining, from step (c)(ii), whether each scanner is finctioning correctly; and
(iv) performing maintenance on any scanner which is determined in step (c)(iii) not to be functioning correctly.

8. The method of claim 7, wherein steps (c)(ii) through (c)(iv) are performed during step (b).

9. The method of claim 8, wherein steps (c)(ii) through (c)(iv) are also performed a plurality of additional times throughout the clinical trial.

10. The method of claim 2, wherein step (d) comprises developing software for analyzing the image data.

11. The method of claim 10, wherein the software comprises software for performing a script-driven analysis.

12. The method of claim 11, wherein the script-driven analysis comprises volumetric analysis and perfusion analysis.

13. The method of claim 12, wherein the software further comprises software for performing automated warp-based registration to align time points in the image data and for performing semi-automated tumor margin identification through geometrically constrained region growth.

14. The method of claim 10, wherein the software comprises software for automatically identifying an arterial input function relating to the tumor.

15. The method of claim 14, wherein the software further comprises software for performing an automated calculation of the parameters.

16. The method of claim 15, wherein the automated calculation of the parameters is performed using a Tofts model.

17. The method of claim 15, wherein the automated calculation of the parameters is performed using a Lee model.

18. The method of claim 10, wherein the software comprises software for producing an electronic audit trail.
